(19) United States
(12) Patent Application Publication
(10) Pub. No.: US 2003/0095962 A1
(43) Pub. Date: May 22, 2003

(54) Chimeric cell-surface receptors that undergo antigen-induced association

(76) Inventors: Hiroshi Ueda, Tokyo (JP); Teruyuki Nagamune, Jawagoe-shi (JP); Hajime Nishimura, Yokohama (JP); Izumi Kumagai, Aoba-ku (JP); Kouhei Tsumoto, Taihaku-ku (JP); Kazuo Todokoro, Abiko-shi (JP); Paula A. Mahoney, Benicia, CA (US); Walt Mahoney, Benicia, CA (US)

Correspondence Address: Roche Diagnostics Corporation, 9115 Hague Road, Bldg. D., P.O. Box 50457, Indianapolis, IN, 46250-0457, US

(21) Appl. No.: 09/898,121

(22) Filed: Jul. 3, 2001

Related U.S. Application Data
(63) Continuation of application No. 09/032,369, filed on Feb. 27, 1998, now abandoned.

(30) Foreign Application Priority Data
Feb. 27, 1997 (JP) 9-44487

(51) Int. Cl. A61K 39/395; G01N 33/567; C07K 16/44

(52) U.S. Cl. 424/130.1; 435/7.21; 530/387.3

(57) ABSTRACT

This invention provides a system for controlling the phenotypic characteristics of cells. A pair of chimeric polypeptides is anchored in the plasma membrane, each of which has a variable region sequence and an effector sequence. The polypeptides are independent in the absence of antigen, but form a stable complex with each other when antigen is provided. This drives the effector sequences together in a manner that produces a receptor activation signal, leading to a phenotypic change. By titrating the amount of antigen present in the environment, the degree of phenotypic change can be regulated. Cells bearing chimeric polypeptides can be used to measure the concentration of antigen or select cells transfected with a therapeutic gene. The chimeric polypeptides can be used as a switch to turn on and off characteristics such as proliferation, apoptosis, degranulation, and protein synthesis, or to target cytolytic cells to antigen-bearing targets

### RELATED APPLICATIONS

This application claims the priority benefit of Japanese patent application JP 9-44487 by Ueda et al. [attorney docket 33746-20055.40], filed Feb. 27, 1997, under provisions of the Paris Convention and Section 119(a) of Title 35 of the U.S. Code. The priority Japanese patent application is hereby incorporated herein by reference in its entirety.

### TECHNICAL FIELD

This invention relates generally to the fields of cell biology, immunochemistry and artificial cell surface receptors. More specifically, it provides a system for obtaining cells with chimeric receptors having variable regions that drive dimerization in the presence of extracellular antigen, creating an intracellular signal that affects cell phenotype.

### BACKGROUND

The ability to control phenotypic characteristics of cells has the potential to expand Most previous techniques to increase or decrease the number of cells in the population, or to elicit particular cellular functions, have been non-specific or have relied on triggering mechanisms already expressed by the cell.

Controlling cell growth is beneficial in improving efficiency of production of useful bioactive substances (Makishima et al.). Bioactive substances can be produced rapidly in the logarithmic growth period by growing cells in the shortest possible time period, then reducing the cell growth speed when and after the number of the produced cells has reached a certain level. This extends the period of production, and allows more substance to be produced by the cells.

Conventional methods for controlling cell growth include adjusting the quantity of cell growth factors, cytokine and other additives in culture media, and varying the concentration of serum. However, cell viability decreases when serum concentration is reduced to suppress cell growth. Another problem is the cost of growth factors and cytokines. To solve these problems, substances to act on the cell growth-related intracellular signal transduction paths have been artificially designed (Crabtree et al.).

A method to control cell growth is to activate or deactivate a growth factor receptor using substances different from the growth factor. Chimeric receptors comprising a growth factor receptor and a receptor of a specific hormone such as estrogen are expressed in the cells. Cell growth is stimulated by binding the growth factor to the extracellular portion of the chimeric receptor or binding the specific hormone to the intracellular portion. An example of the latter is a chimeric receptor consisting of the granulocyte colony-stimulating factor receptor and the hormone binding region of estrogen receptor (Ito et al).

Chimeric receptors are also an effective means to select cells introduced with foreign genes. Conventionally, a drug resistance gene or a marker gene is introduced into cells together with a therapeutic gene and transfected cells are selected using the signals of those selective genes. The therapeutic gene-containing cells may not proliferate, and the effect of gene therapy may be extremely low. However, a vector can be used encoding a chimeric receptor (for example, a growth factor receptor fused with a hormone receptor) along with the foreign gene. After introducing the vector into the cells, it is possible to expand the target cells by culturing in the presence of specific hormones thus increasing the proportion of cells with the foreign gene. This approach allows ex vivo cell selection and expansion, and in vivo selective growth of cells expressing the therapeutic gene (Ito et al.). Receptor constructs have also been prepared in which antibody binding sites are part of a molecular chimera.

Ueda et al. (1992) constructed artificial chimeric cell-surface receptors, combining murine IgM with the cytoplasmic portion of a human growth factor receptor. The chimeric receptor showed both antigen binding and protein tyrosine kinase activity, but the kinase activity was constitutive and independent of antigen binding. With IgM lacking the C_{H2} domain, autophorphorylation increased with increasing concentrations of hapten-BSA conjugate. Monovalent hapten could not induce phosphorylation, but inhibited stimulation by the conjugate.

U.S. Pat. No. 4,859,609 (Dull et al.) constructed hybrid receptors that comprise the ligand binding domain of a predetermined receptor, and a heterologous reporter polypeptide. The ligand binding domain (something other than an immunoglobulin) undergoes a conformational change upon binding of the ligand, which in turn affects the reporter peptide attached on the C-terminal end. The model reporter molecule is a phosphorylkinase. An assay method is claimed, in which the hybrid receptor is incubated with a test sample, and then a conformational change is correlated with the presence of ligand in the sample.

International Patent Application WO 96/23881 relates to chimeric receptors for regulating cellular proliferation and effector functions. The proposed chimeric receptors are various combinations of an extracellular clustering domain, a transmembrane domain, an intracellular clustering domain, an intracellular proliferation signaling domain (that signals a host cell to divide), or an intracellular effector domain (that signals an effector function). Proliferation signaling can be taken from Janus tyrosine kinases, or from receptors from interleukins such as IL-2.

Bach et al. outlines experiments in which chimeric receptors with antibody specificity were introduced into mast cells. The receptors comprised a single-chain Fv of an antibody molecule, fused either to a chain of an Fc receptor for IgE, an Fe receptor for IgG, or a chain from the CD3 complex. The antibody was specific for the hapten TNP. Triggering of the cells with a TNP-protein conjugate resulted in degranulation.

Kruskal et al outlines experiments with the high-affinity Fc receptor (CD64) and the mannose receptor, two receptors that mediate phagocytosis. When transfected into Cos cells which are normally non-phagocytic, only the mannose receptor permits the cells to ingest the corresponding ligand. However, a fusion protein comprising the ligand binding domain of CD64 and the intracellular domain of the mannose receptor permitted the cells to phagocytose antibody-opsonized erythrocytes. The chimeric receptor performed this function better if the membrane spanning region was taken from the mannose receptor rather from the CD64.

International Patent Applications WO 95/02686 (Seed et al.) and WO 96/26265 (Seed et al.) relate to chimeric receptors with tyrosine kinase activity. Model ligand binding domains are taken from CD16, and the intracellular tyrosine kinase domain is modeled on members of the Syk family. Cells transfected with these chimeric receptors are reported to be capable of specifically recognizing and destroying a target cell by cytolysis.

U.S. Pat. No. 5,712,149 (Roberts et al.) and International Patent Application WO 97/35004 (Hawkins et al.) relate to chimeric receptor molecules based on CD28. The extracellular domain in CD28 is replaced in U.S. Pat. No. 5,721,149 with that of CD4 or CD8. In WO 97/35004, it is replaced with a single-chain variable region to a hapten like NIP or phOx. It is proposed that cells like cytotoxic lymphocytes or NK cells that normally receive a second signal for activation via the CD28 receptor be modified with these chimeric receptors, so that the second signal can be provided via an alternative ligand. Similarly, European Patent EP 517,895-B1 relates to chimeric receptor molecules comprising an extracellular domain of CD4 or CD8, and an intracellular domain with tyrosine kinase activity from either a T cell receptor or the high affinity receptor for IgE.

International Patent Application WO 95/02684 relates to materials for inducing programmed cell death by receptor dimerization. The application claims DNA constructs encoding a domain capable of binding small ligands such as cyclosporin A, tetracycline or steroids, and also encoding a domain capable of initiating apoptosis.

Hess et al. provide a cytotoxic chimeric receptor comprising the extracellular domain of CD40 and the intracellular domain of the P55 tumor necrosis factor receptor. This hybrid retained the biological activity and the ligand specificity of the wild-type receptor domains when expressed in three different cell lines. Activation with either anti-CD40 antibody or the CD40 ligand in soluble or membrane-bound forms induced death of the host cell expressing the receptor.

Research in another area relates to the immunochemical properties of antibody fragments. New discoveries relating to isolated variable region polypeptides are related in European Patent Application EP 816,850, and in Ueda et al. (1996b). Variable region polypeptides from the heavy and light chain region of specific antibody molecules can be produced that normally stay apart in solution, but come together in the presence of antigen. The property of antigen-driven association can be exploited to measure the concentration of antigen in a test sample—either in a sandwich type separation assay, or in a homogeneous assay in which at least one of the polypeptides is provided with a reporter molecule such as fluorescein.

### SUMMARY OF THE INVENTION

The invention described in this disclosure provides a system for controlling the phenotypic characteristics of cells. A pair of chimeric polypeptides is anchored in the plasma membrane, each of which has a variable region sequence and an effector sequence. The polypeptides are independent in the absence of antigen, but form a stable complex with each other when antigen is provided. This drives the effector sequences together in a manner that produces a receptor activation signal, leading to a phenotypic change. By titrating the amount of antigen present in the environment, the degree of phenotypic change can be regulated. for a variety of research, diagnostic, and therapeutic purposes.

Certain embodiments of the invention relate to a pair of chimeric polypeptides consisting of a first polypeptide and a second polypeptide that complex with each other in the presence of an antigen. Each polypeptide of the pair comprises an extracellular domain comprising a variable domain sequence, a transmembrane domain, and an effector sequence. Upon expression of the two polypeptides in a host cell, the pair of chimeric polypeptides has the property that contacting the cell with an analyte promotes association of the variable domain sequence of the first polypeptide with the variable domain sequence of the second polypeptide, thereby promoting association of the effector sequence of the first polypeptide with the effector sequence of the second polypeptide, inducing a change in cell phenotype. Preferred variable region sequences are complementary V_{H} and V_{L} sequences. Effector sequences of interest are homologous to subunits of a proliferation receptor, a degranulation receptor, a cytotoxic receptor, a phagocytic receptor, or an apoptosis receptor, and participate in generating a phenotypic change of interest upon dimerization in the cell where it is normally found.

Further embodiments are host cells expressing chimeric polypeptides of this invention. Exemplary cells are those that are stimulated by the antigen to undergo cell division at an increased rate. Also exemplary are cells that are stimulated by antigen to degranulate, phagocytose or lyse a target particle, or undergo apoptosis.

Other embodiments of this invention relate to two polynucleotide sequences respectively encoding the first and the second polypeptides of the pair of chimeric polypeptides comprised in either a single polynucleotide or in two separate polynucleotides. Also embodied are expression systems comprising the sequences in a common vector or in two separate vectors.

Further embodiments relate to methods for obtaining the chimeric polypeptides or host cells of this invention, comprising introducing into a host cell an expression system encoding the polypeptides, contacting the cell with the antigen, and selecting cells expressing the desired phenotype. Another embodiment is a method for selecting cells transformed with yet another polynucleotide sequence, by including the polynucleotide sequence in the expression system for the chimeric polypeptides, and selecting cells expressing the phenotype regulated by the effector sequences of the polypeptides, such as growth rate.

Additional embodiments of the invention relate to changing the phenotype of a cell in culture or in vivo by contacting a cell expressing the chimeric polypeptides with the antigen. This can be performed, for example to increase proliferation, reduce the number of cells by apoptosis, or stimulate phagocytosis or cytolysis of a target.

Other embodiments relate to pharmaceutical compositions comprising an effective amount of a polypeptide, polynucleotide, or cell of this invention. Also embodied are the use of polypeptides, polynucleotides or cells in the preparation of a medicament for the treatment of a human or animal body by surgery or therapy.

Further embodiments of the invention will be apparent from the description that follows.

### BRIEF DESCRIPTION OF THE FIGURES

FIG. 1 is a diagram of a model for a pair of chimeric receptor polypeptides according to the invention in the associated configuration. Two different views are shown. The top half of each view represents the predicted structure of the V_{H} and V_{L} regions from HyHEL-10, based on the known crystal structure of the HyHEL-10 Fab fragment. The distance between the terminal Alanine (113) of V_{H} and the terminal Lysine (107) of V_{L} is 43.0 Angstroms. The lower half of each view represents the predicted structure of a homodimer consisting of the D2 domain of the human receptor for erythropoietin (“EPD2”). This domain connects the Epo binding region with the transmembrane and intracellular domains of the intact receptor. The N-terminal ends are near the outer limit of the associated dimer, and separated by a distance (35.9 Angstroms) that closely matches that of the variable region C-terminal ends. When V_{H} and V_{L} are fused to the erythropoietin receptor beginning at this position in the D2 domain, association of V_{H} with V_{L} can drive the D2 domains together, which in turn can drive the intracellular domains together thereby generating kinase activity. To provide restriction sites and facilitate preparation, short linker sequences of a few amino acids were introduced between the variable region sequences and the EpoR sequences in the genetic constructs.

FIG. 2 is a half-tone reproduction of a Western blot, showing the measurement of the expressed proteins of the Ba/HEL cell which were produced according to Example 2. Ba/F3 cells were introduced with vectors alternately encoding V_{H} and V_{L} sequences of an anti-hen egg lysozyme (HEL) antibody fused with the transmembrane and intracellular domain of the mouse EpoR receptor (82% identical to the human EpoR). Clone colonies were then expanded in the presence of the antigen HEL. The blot was obtained using a Triton™ X-100 lysis of the clones listed above each lane, or untransformed Ba/F3 cells. It was developed using a first antibody against the intracellular domain of the EpoR followed by labeled anti-immunoglobulin. The arrow shows a band corresponding to about 65 kDa, present in each of the transformed clones. This corresponds to the expected molecular weight of the two variable region-receptor fusion polypeptides.

FIG. 3 is a graph showing the relation between the concentrations of antigen n a culture medium and cell growth rates. Clone G8 was obtained by transforming Ba/F3 cells with the V_{H}/V_{L} EpoR vector constructs and growing in the presence of antigen. In this experiment, separate cultures of Clone G8 was cultured with different constant amounts of the antigen HEL over a 5-day period. Cell proliferation was dose-dependent, and was inducible up to at least about 5 times the rate by including antigen in the culture medium.

### DETAILED DESCRIPTION

This invention provides chimeric polypeptide pairs that are expressed on a cell membrane. The two polypeptides having the property that they associate with each other when a particular antigen is present As a result of the antigen-induced association, effector sequences come together in a way that creates an intracellular signal, which in turn affects the phenotype of the cell in a desirable fashion.

Each of the two constituent members of the polypeptide pair comprises the following elements:

a driver of the complexation reaction on the extracellular portion of the polypeptide. The driver is a variable domain sequence, with the property that the presence of antigen promotes the formation of a stable complex with the opposing variable domain on the opposing peptide.

a reporter of the complexation reaction, termed the effector sequence, which does not associate in an activated configuration with the effector on the opposing peptide unless the variable sequences are complexed, and which produces an intracellular signal when complexed with the opposing effector sequence, possibly in combination with other intracellular elements

a transmembrane or membrane insertion domain, that keeps the polypeptide anchored in the membrane, permitting it to diffuse along the lipid bilayer and complex with the complementary peptide in the presence of antigen.

The chimeric polypeptides of this invention can be expressed in a host cell to instill the cell with antigen-activatable effector function. The polypeptides can be used as a switch, to turn on the effector function by adding antigen to the cell, or turn off effector function by removing antigen. The polypeptides can also confer targeting ability of both the cell and the effector function for targets bearing antigen, or can be used to bypass certain second signal requirements of the immune system. Applications for host cells of this invention is described in detail at a later point in the disclosure.

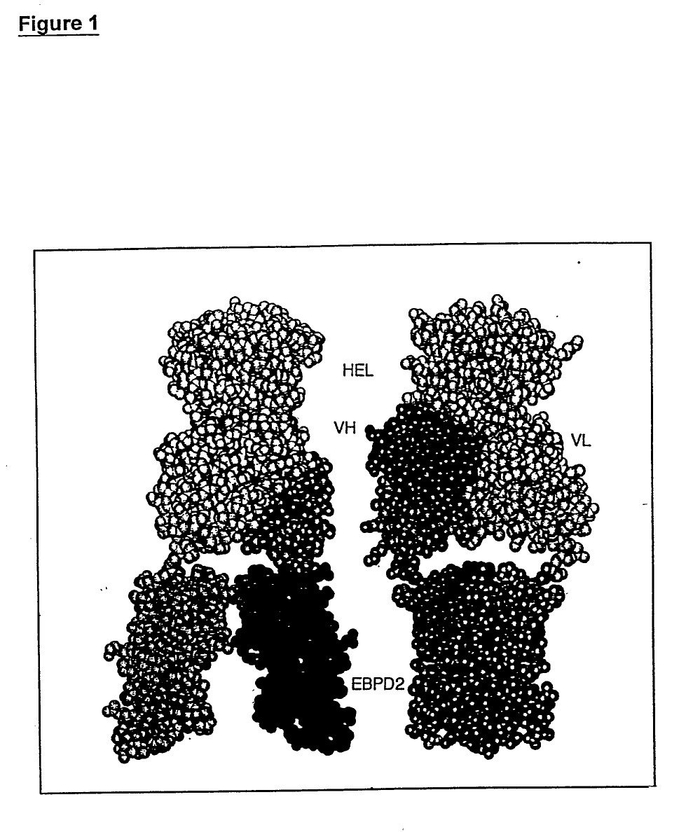

A portion of an exemplary chimeric receptor of this invention is shown in FIG. 1. V_{H} and V_{L} regions of a specific antibody are each attached to identical fragments of the receptor for erythropoietin (EpoR) at a point where the spanning distance between the C-terminal residues of associated V_{H} and V_{L} sequences matches that of the N-terminal residues of the receptor fragments. Just the extracellular portion of each receptor fragment is shown. Towards the bottom of the diagram would be the cell membrane, and the receptor fragment would extend through the membrane to the intracellular domain The intracellular or cytoplasmic domain of the EpoR contains a JAK kinase, which, upon dimerization of the polypeptide chains activates tyrosine kinase activity. Ba/F3 cells expressing this pair of chimeric polypeptides can be induced to proliferate by supplying antigen in a dose-dependent fashion.

The following disclosure further describes the mailing and using of the invention.

The skilled artisan has several strategic choices for the preparation and assembly of the polypeptides of the invention. Generally, the polypeptides are obtained by a method that includes the following steps, which will each be discussed in turn.

selecting two variable domain sequences that form a complex that is stabilized in a solution if the solution contains the antigen of interest;

selecting two effector sequences, based on a receptor complex having at least two identical or non-identical polypeptide subunits, wherein activation of the receptor involves association of the two effector sequences;

preparing fusion peptides each containing one variable domain sequence, one effector sequence, and one transmembrane sequence; and

confirming that the two fusion peptides have the desired functional properties when expressed in a suitable host cell.

The two variable domain sequences are usually V_{H} and V_{L} domains, although other combinations are possible (for example, homologous or heterologous V_{L}-V_{L} pairs in the Bence Jones configuration, and T cell variable region pairs, especially Vγ-Vδ pairs). The variable domain sequences may correspond to a complete intact variable region domain, or may be longer and shorter in length, or incorporate amino acid changes, inserts, or deletions. Typically but not invariably, each variable domain will have the three CDR regions found in intact variable region. Sensitive to alterations are segments that make up the antigen binding site and the interface between the variable region pair, and changes should be made so as not to impair the required binding properties. The variable region domains can be of human origin, mouse origin, or of any other species, or they can be artificial sequences designed as a chimera or consensus of multiple species. Variable regions of human origin (or having human framework residues) are of interest for therapeutic applications, in order to minimize unwanted immunogenicity. Also of interest are variable regions of camel origin, or variable regions modified to incorporate camelid mutations which decrease the affinity between variable regions.

The “antigen” to which the variable region pairs bind can be a small molecule drug or hapten, or a protein, nucleic acid, carbohydrate, proteoglycan, glycolipid, or any structure which can be used to select the variable region pairs or binds the variable region pair with sufficient affinity and specificity. An antigen which induces the dimerization of a variable region pair is also referred to as a “driver antigen”.

Raising and selecting variable regions with the specificity for a particular antigen is standard practice in the art. General techniques used in raising, purifying and modifying antibodies, and the design and execution of immunoassays, are found in Handbook of Experimental Immunology (D. M. Weir & C. C. Blackwell, eds.); Current Protocols in Immunology (J. E. Coligan et al., eds., 1991); David Wild, ed., The Immunoassay Handbook (Stockton Press NY, 1994); and R. Masseyeff, W. H. Albert, and N. A. Staines, eds., Methods of Immunological Analysis (Weinheim: VCH Verlags gesellschaft mbH, 1993). For hybridoma technology, the reader is referred generally to Harrow & Lane (1988), U.S. Pat. Nos. 4,491,632, 4,472,500, and 4,444,887, and Methods in Enzymology, 73B:3 (1981). Briefly, the immunogen is optionally modified to enhance immunogenicity, for example, by aggregating with glutaraldehyde or coupling to a carrier like KLH, and then mixed with an adjuvant, preferably Freund's complete adjuvant for the first administration, and Freund's incomplete adjuvant for booster doses. The most common way to produce monoclonal antibodies is to immortalize and clone a splenocyte or other antibody-producing cell recovered from an animal that has been immunized. The clone is immortalized by a procedure such as fusion with a non-producing myeloma, by transfecting with Epstein Barr Virus, or transforming with oncogenic DNA. The treated cells are cloned and cultured, and clones are selected that produce antibody of the desired specificity. Specificity testing is performed on clone supernatants usually by immunoassay.

Other methods for obtaining specific variable regions from antibodies or T cells involve contacting a library of immunocompetent cells or viral particles with the target antigen, and growing out positively selected clones. Immunocompetent phage can be constructed to express immunoglobulin variable region segments on their surface. See Marks et al., New Engl. J. Med. 335:730, 1996; WO patent applications 94/13804, 92/01047, 90/02809; and McGuinness et al., Nature Biotechnol. 14:1149, 1996. Phage of the desired specificity are selected by adherence to antigen attached to a solid phase, and then amplified in E. coli.

Screening variable regions with the property of antigen-dependent association involves assaying the association of one of the variable regions with the other in the presence and absence of antigen. Solid phase enzyme or fluorescein labeled association tests are quite appropriate, and fully described in E.P. patent application E.P. 816,850 (Ueda et al.), which is hereby incorporated herein by reference in its entirely. The association between heavy and light chains is due in large part to association between C_{H1} and C_{L}. It is estimated that about 1 in 10 variable region pairs have sufficiently low association constant when detached from the constant regions for use in this invention without further association. The association constant is predicted to be a function of interacting residues along the interface. Accordingly, variable domains that have an antigen-dependent association can be obtained for any antigen, using this selection strategy. Prototype antigens in the development of this invention have included hen egg lysozyme and digoxin.

The selected variable region pair should have an association constant of one variable region for the other should be at least 10-fold higher in the presence of antigen, and is progressively more preferred if it is at least about 10², 10³, 10⁴, or 10⁵ fold higher. Association in the absence of antigen is generally less than 10⁸ M⁻¹ and preferably less than 10⁶ M⁻¹. Association of the variable regions for each other in the presence of antigen, and association of antigen for the variable region complex, is generally over 10⁸ M⁻¹, preferably above 10¹⁰ M⁻¹, and more preferably above 10¹² M⁻¹. Association constants can be modified, if desired, by altering amino acids along the interface. It is not necessary to measure the affinities to practice the invention, as long as a sufficient difference is observed in the presence or absence of antigen in the intended context

The effector sequences attached to each of the variable region sequences in the polypeptide pair have the property that they promote a desirable phenotype of the cell under conditions where they are driven together by antigen-induced association of the variable region sequences.

Most often, the effector sequences are taken and adapted from a receptor complex having at least two polypeptide subunits, with the property that the polypeptide subunits promote the desired feature upon dimerization of the subunits. The association may form a homodimer (where the subunits are identical) or a heterodimer (where the subunits are different). The dimerization typically occurs upon binding of a ligand to an extracellular component of the receptor; for example, as a result of a conformational change induced by binding, or because multiple ligands induce subunit aggregation, or because the ligand is polyvalent and binds to each subunit, bringing them together. Upon formation of a stable dimer, an intracellular signal is produced which leads to expression of the desired phenotype by the cell. The dimerizing subunits are then adapted for this invention by removing the binding site(s) for the natural ligand and substituting the variable region sequences, one on each subunit.

There are three criteria for the selection of a receptor from which to take the effector sequences: the biochemistry of activation, the spatial orientation of the activated complex, and the effect of activation on the host cell.

The biochemistry of activation of the prototype receptor involves in some aspect the dimerization of the subunits from which the effector sequences are taken. The mechanism by which dimerization affects cell phenotype need not be known in detail, providing the desired result is obtained. A number of cell surface receptors provide an intracellular signal by tyrosine kinase activity: for example, the chains of the EpoR comprises elements that generate kinase activity upon dimerization, while dimerization of certain Fc receptor subunits leads to association with a second polypeptide with kinase activity. Either types are suitable for use in this invention. However, chimeric polypeptides of this invention based on the latter example will only work in the expected fashion in cells expressing the second polypeptide, either constitutively or by cotransfection. In addition, different cells may have different responses to a similar intracellular signal; for example, one cell undergoing degranulation and another cell undergoing phagocytosis. The host cell is chosen, in part, as being able to deliver the desired phenotypic change upon receptor activation.

The spatial orientation of the prototype receptor is a relevant consideration, because it is usually necessary that the variable regions of the chimeras be able to drive the effector sequences together in a similar orientation as in the native receptor. Accordingly, the variable sequences in the associated configuration must be joinable to the effector sequences in the activated configuration. Effective combinations can be determined empirically, but it is helpful if crystal structure data is available for the candidate receptor prototype. If so, then the candidate can be examined to determine whether there is a site in the extracellular domain where the spanning distance between the receptor chains matches the spanning distance of the associated variable regions. A candidate receptor is not necessarily excluded if such a site does not exist, since there are other ways of achieving the connection; however, receptors with matching spanning distances just outside the cell surface are generally preferred.

The effect that activation of the effector regions will have upon the host cell is selected depending on the ultimate use of the cell bearing the chimeric polypeptides.

One possible effect is to initiate or accelerate proliferation of the host cell. Suitable for use as model receptors are those that stimulate growth in response to a particular ligand, such as a growth factor or hormone, a cytokine, a foreign antigen, or a stimulatory surface protein on another cell. Receptors activated by the formation of homodimers include those for erythropoietin, human growth hormones, epidermal growth factors, and platelet-derived growth factors. Receptors activated by the formation of heterodimers comprising non-identical subunits include, for example, receptors for certain cytokines such as GM-CSF.

Also of interest are the β and γ subunits of the IL-2 receptor. Also of interest are receptors comprising one or more of the family of Janus kinases, i.e., JAK1, JAK2, JAK3, Tyk2 and PTk-2. Also of interest are HER2/neu, HER3/c-erbB-3, the insulin receptor, insulin-like growth factor receptor, M-CSF receptor, c-kit, FGF-receptor, and the Trk family of tyrosine kinases, including the NGF receptor and ROR1,2.

Another possible effect is the simulation of granule release or protein synthesis. One of the first receptors proven to be activated by dimerization is the high-affinity receptor for IgE on mast cells and basophils (FcεRI). The sequence of the human IgE receptor is disclosed in U.S. Pat. No. 5,639,660 (Kinet et al.). Suitable candidates for providing the effector sequences of a chimeric receptor is the γ chain of the FcεRI receptor, the CD16α chain of the FcγRIII receptor, and the ζ chain of the CD3 receptor complex. In mast cells, inducing chimeric receptors made up of variable region sequences and these effector sequences may induce granule release, measurable as hexoseamidase activity. In macrophages and NK cells, inducing variable region FcγRIII receptors may induce cytokine production and expression of certain cell surface markers. Suitable subunits and regions of these receptors are provided in Bach et al.

Another possible effect is stimulation of phagocytosis or cytotoxic lysis of a neighboring particle or cell. Particularly suitable as a model effector sequence for phagocytosis is the mannose receptor (Kruskal et al.). Both the intracellular and the transcellular domain of the mannose receptor are used, alternately replacing the mannose-binding ectodomain with the variable region sequences. Similarly, receptors that mediate cytotoxicity (T cell receptors, Fe receptors in cells mediating antibody-dependent cellular cytotoxicity, and receptors on NK cells) can have their usual ligand binding domains substituted with variable region sequences that undergo antigen-induced association. Suitable receptors include members of the Syk kinase activity. Suitable receptor subunits include ZAP-70 and either Fyn or Lek. Suitable effector sequences from these receptors are described in WO 95/02586 (Seed et al.) and WO 96/26265 (Seed et al.).

Other types of immune effects are also contemplated. Stimulation of certain cells of the immune system in certain ways involve a plurality of different signals conveyed to the cell through different receptors. Chimeric polypeptides according to this invention can be substituted in one of the signaling pathways, changing the nature of the second signal while leaving the specificity requirements of the other pathway intact. In one example, the cytoplasmic and preferably also the transmembrane and a portion of the extracellular domain are used, replacing the N-terminal 108 amino acids or so with variable region sequences that undergo antigen-induced association. For suitable CD28 fragments, the reader is referred to U.S. Pat. No. 2,712,149 (Roberts) and WO 97/35004 (Hawkins et al.). In another example, ζ chain of the T cell receptor may be suitable as an effector for early activation of T lymphocytes, detectable by induction of CD69 expression.

Yet another possible effect is induction of apoptosis. Effector sequences can be based on the family of cell death receptors, members of which include the Fas receptor, TNF receptor I, death receptor 3 and 4 (DR3 and DR4), and the cytopathic avian receptor 1 (CAR1). An artificial receptor made up of the extracellular and transmembrane domain of CD40, and the intracellular domain of the p55 TNF receptor is a strong initiator of apoptosis when transfected into a number of different cell lines, upon induction by dimerization (Hess et al.). To obtain a polypeptide pair according to this invention that is capable of initiating apoptosis upon presentation of antigen, the CD40 extracellular domain of this construct or the extracellular ligand binding domain of another cell death receptor is alternatively substituted with each of the two variable region sequences.

Of course, the exact amino acid sequence of the effector fragments used in the chimeric polypeptides of this invention need not be exactly identical to that of the native receptor, providing the same effector function is achieved. Complete identity will generally help ensure the function is faithfully reproduced, but it may be desirable to make certain adjustments to improve functionality, adjust spanning distances or interface interaction, reduce immunogenicity, or for other purposes. Typically, the effector sequence will have a degree of identity with a sequence in the native receptor subunit of the order of at least about 70%, and preferably at least about 80% or 90%. Identity is calculated as the percent of amino acids in the consecutive sequence of the native molecule that are preserved in the same order (with no penalty for gaps or inserts) in the sequence of the chimeric polypeptide. Where the native enzyme is made up of non-identical fragments or subunits, the polypeptide pair will usually also have non-identical effector sequences. Where the native receptor is made up of identical fragments or subunits, the polypeptide pair can have identical or non-identical effector sequences, independently of the variable region sequences, which are usually non-identical. For instances where the effector sequences are non-identical, the choice of which effector sequence to attach to which variable domain sequence may make little difference, or can be optimized by empirical testing.

Once variable region sequences and effector sequences have been chosen, the chimeric polypeptides are designed to incorporate the necessary features in a workable arrangement.

A transmembrane domain is included to keep each of the chimeric polypeptides anchored in the membrane and permit lateral movement in the lipid bilayer. The term “transmembrane” domain means that the domain is sufficiently lipophilic to penetrate the lipid bilayer and keep the entire polypeptide inserted in the membrane in a stable fashion. Typically, the transmembrane domain will span the entire lipid bilayer one or more times. The ability of a domain to keep a protein anchored in the membrane is verified empirically, but is predictable on the basis of a high proportion of lipophilic residues and relatively few charged residues, based on known algorithms.

Most frequently, the transmembrane domain will be present in the chimeric polypeptides between the variable region sequences and the effector sequences. However, it is recognized that some effector functions are generated in the extracellular domain, or any combination of domains inside and outside the cell and in the lipid membrane. The order of each of the functional elements in the chimeric polypeptides will reflect

As a general rule, the transmembrane domain will be derived from the same receptor prototype as the effector sequence, since it is generally best adapted to convey the ligand binding signal across the cell membrane, and may even participate in providing the second signal. However, there are exceptions in which substitution of a heterologous transmembrane domain improves activity: see U.S. Pat. No. 5,641,863 (Schreiber et al.). Third party or artificially designed polypeptide sequences can be used for transmembrane domains in this invention where appropriate. Another reason to use a different transmembrane domain is to help solve the issue of how to connect the variable region sequences with the effector sequences. Elsewhere in this disclosure, it is demonstrated that the spanning distance of the EpoR subunits is well matched to that of variable region sequences, permitting them to be driven together when the variable region sequences associate. Other receptors with spanning distances that do not match associated variable regions outside the cell can be used with this invention may still be suitable candidates if they match associated EpoR subunits inside the cell—providing the intracellular domains of the candidate receptors are sufficient to provide the activation signal. The candidate construct would comprise a variable region sequence of desired antigen specificity, linked to the extracellular D2 domain and transmembrane domain of EpoR, linked to the intracellular domain of the new receptor.

The most usual configuration of the chimeric polypeptides is for the C-terminus of each variable region to be linked to the N-terminus of each effector, since most receptors are oriented with the N-terminal outwards. However, other configurations are possible and may be desirable where the receptor is oriented C-terminal outwards. It is possible to trim a few residues from the variable regions to enhance the match of the spread when the components are in the associated configuration. It is also possible to have a linker sequence between the variable sequence and the sequence based on the receptor prototype on one or both chains. Preferred linker sequences are less than about 10 amino acids in length, and where linkers of more than a few amino acids are used, it is appropriate to begin with candidates that form a rigid bridge, such as an a-helix. This will help ensure that the variable region sequences will be able to drive the effector sequences together so as to produce the signal for activation.

A chimeric polypeptide of this invention is typically prepared by expressing a recombinant polynucleotide encoding it. Encoding sequences for the various elements are linked in a recombinant or synthetic polynucleotide, prepared from known sequence data or polynucleotide templates according to standard techniques of genetic engineering. To have the encoding sequences expressed in a host cell, an expression vector is prepared in which the sequences are operatively linked to transcription and translation control elements appropriate for the host cell. To express the two complementary chimeric polypeptides in the same cell, the expression system can either be provided as a single expression vector comprising encoding sequences for each of the two polypeptides, or separate expression vectors for each of the polypeptides. The expression vectors are introduced into the cell using any method known in the art, including but not limited to electroporation, calcium phosphate precipitation, or contacting with a polynucleotide-liposome complex; or (where the vector is a viral vector) by transduction or infection. Where the chimeric receptors comprise a effector sequences from growth factor receptors, successfully transfected cells can simply be grown out of the cell population by culturing in the presence of antigen. Otherwise, a selectable marker for transfection is included in the vector system, or cell colonies are identified (usually after cloning) by immunochemical identification of the encoded protein or by functional testing.

The success of the constructs is determined empirically by contacting the cells with the antigen and observing whether this successfully promotes the desired phenotypic change. Different flasks of cells are cultured for a time under conditions that permit expression of the chimeric peptides, and in the presence or absence of antigen. The flasks are then compared using a standard assay appropriate for the desired phenotype; for example, by observing cell growth or apoptosis, measuring protein expression or secretion by immunoassay, or observing phagocytosis or lysis of target cells or particles bearing the antigen.

Once a pair of chimeric polypeptides with all the desired properties has been obtained, it can serve as a prototype for other polypeptides. The fold of immunoglobulin variable region domains is consistent between variable regions of different specificity. Accordingly, one variable region domain can be substituted for another atop the effector sequence. Alternatively, the CDRs of a variable region of a new specificity can be cassetted into the framework of a proven fusion polypeptide, taking care to avoid disturbing the association properties of the variable domain interface.

The chimeric polypeptide pairs of this invention have a number of applications in both clinical medicine and research. Three applications of particular interest are as adapted host cells for laboratory production, therapeutic use and as assay reagents.

Use of this invention in laboratory research and production is appropriate whenever it is desirable to have the ability to control the phenotype of a cell by titrating the amount of an antigen in the culture medium. Depending on the nature of the effector sequence, antigen can be added, for example, to increase the rate of cell proliferation, to make T cells receptive to an immunogen, or to remove certain cells in a mixed population.

The chimeric polypeptides also provide a method of selecting out cells that have been successfully transcribed with another encoding sequence of interest, such as a gene needed for gene therapy. The host cell is introduced with an expression system for the chimeric polypeptides having a growth factor effector function; either one vector encoding both polypeptides or separate expression vectors encoding each one. The gene is included in one of the expression vectors. After transfection, cells are cultured to permit expression of the vectors in the presence of the antigen. For example, if the cell is a Ba/F3 cell and the culturing is performed in the presence of antigen and without IL-3, then the cells that grow will be those that have been successful transfected with the chimeric polypeptides, and hence the gene of interest.

Use of the products of this invention in therapy has a number of aspects. In one aspect, this invention provides a switch, whereby a cell administered in therapy can be controlled after administration in vivo. Typically, the antigen binding activity is chosen so that an inert hapten can be administered to the patient to trigger the phenotypic change, such that the hapten has no direct effect on the patient, and other substances the patient is likely to encounter will not cross-react. Suitable antigens of this type include inactive analogs and enantiomers of naturally occurring substances or simple chemical structures. For example, previously administered stem cells having chimeric polypeptides with growth factor effector sequences can be induced to proliferate by giving antigen to the patient to stimulate regeneration at critical times. Lymphocytes having a CD28 receptor chimera can be preactivated at a local site by giving the antigen locally.

This invention also provides a method by which cells administered to a patient for therapy can be eliminated after their task is complete. For example, it may be desirable at some time to remove third-party donor cells administered during regeneration, or genetically engineered killer cells administered to fight an infection or a tumor. Before administration, the cells are equipped with chimeric polypeptides of this invention comprising effector sequences for apoptosis. The cells are then administered to treat the condition in the patient. Once the patient recovers, the antigen is given systemically. This induces apoptosis in the administered cells, reducing any future risk of disease emanating from the cells themselves.

Another therapeutic use provided by this invention is a new method for targeting a therapeutic effect. For example, phagocytic or cytotoxic cells can be equipped with chimeric polypeptides with antigen specificity for the target, and effector sequences for the respective function of the cells. Thus, cytotoxic cells can be both targeted and activated for tumor cells if the variable region sequences are specific for a tumor antigen. In a variation of this approach, the variable region sequences are developed against a hapten, and the patient is also administered with an effective anti-tumor antibody (for example, a stabilized single-chain variable region) conjugated with the hapten. The antibody first paints the tumor with the hapten, and then the cell binds and initiates cytolysis by way of the chimeric receptor polypeptides. In another aspect, a cytotoxic cell is equipped with chimeric polypeptides specific for the target site, and having an effector domain for proliferation. The cells are thus equipped to preferentially proliferate in the local milieu near the target cell.

The preparation of compositions for therapeutic use is conducted in accordance with generally accepted procedures for the preparation of pharmaceutical preparations. See, for example, Remington's Pharmaceutical Sciences 18th Edition (1990), E. W. Martin ed., Mack Publishing Co., PA. Pharmaceutical preparations suitable for human use are sterile and substantially free of mycobacteria. For systemic distribution, administration is typically intravenous or intramuscular, although other routes are possible; or the composition can be administered locally near the site of the intended effect.

The chimeric polypeptides of this invention can also be used for the determination of antigen in a sample. The polypeptide pair will have a variable region specific for the antigen to be detected or quantified. Upon activation, the effector sequences will cause the host cell to express a phenotype which is measurable for correlation with the presence or amount of antigen in the original sample. For example, if the activated effector stimulates granule release or peptide synthesis, then the cells can be contacted with the sample for a suitable time to generate the effect. The cell supernatant can be assayed for the presence of one of the secreted components, and the level of the component is correlated with the amount of antigen in the original sample. In another example, if the activated effector stimulates increased cell proliferation, then the cells can be contacted with the sample, and the rate of growth of the cells can be correlated with the amount of antigen in the sample. This is illustrated in Example 4.

Any of the assay embodiments of this invention may be employed for research or diagnostic use. Suitable biological or clinical samples include but are not limited to tissue culture media, urine, plasma, serum, and histological sections. Suitable antigens for detecting or quantifying in a clinical setting include those that correlate with certain clinical conditions, such as ferritin, prostate specific antigen, alpha fetoprotein, carcinoembrionic antigen, hCG, prolactin, thyroid stimulating hormone, progesterone, T3 and T4, free T3 and T4, aldosterone, insulin, and so on. Also suitable are drugs administered in therapy or drugs of abuse.

Polypeptides, cells, vectors and other products of this invention can be packaged in kit form to facilitate distribution. The reagents are provided in suitable containers, and typically provided in a package along with written instructions relating to their use.

The practice of certain aspects of the invention will employ conventional techniques of molecular biology, genetic engineering, microbiology, cell biology, biochemistry and immunology, which are within the skill of the art. These techniques are explained fully standard textbooks, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

All patents, patent applications, articles and publications mentioned herein, both supra and infra, are hereby incorporated herein by reference.

Further illustration of the development and use of chimeric polypeptides according to this invention are provided in the Example section below. The examples are provided as a further guide to a practitioner of ordinary skill in the art, and are not meant to be limiting in any way.

### EXAMPLES

### Example 1

### Preparation of Vector DNA for the Expression of Chimeric Receptor

A vector DNA containing the recombinant gene consisting of V_{H} and V_{L} genes of the antibody HyHEL-10 specific for hen egg lysozyme and mouse erythropoietin gene. Using the vector plasmid pKTN2 (Tsumoto, K. et al., J. Biol. Chem., 269: 28777-28782, 1994) which encodes pelB signal peptide sequence upstream of the structural genes for V_{H} and V_{L} of the antibody HyHEL-10 as a template, V_{H} and V_{L} gene fragments were amplified by the PCR method. The primers used for the PCR amplification were the oligonucleotide having the nucleotide sequence of SEQ ID No. 1 (V_{H}HelSseBack: 5′-primer), and the oligonucleotide having the nucleotide sequence of SEQ ID No. 2 (V_{H}HelAflFor: 3′-primer) for the V_{H} gene fragment, and the oligonucleotide having the nucleotide sequence of SEQ ID No. 3 (V_{L}HelPstBack: 5′-primer), and the oligonucleotide having the nucleotide sequence of SEQ ID No. 4 (V_{L}HelAflFor: 3′-primer) for the V_{H} gene fragment. The 3′-primer was designed to encode a splicing donor sequence, and a restriction enzyme site for linking with the vector was incorporated to each primer. The PCR-amplified V_{H} and V_{L} gene fragments were cleaved with SseI and HindIII, and with PstI and HindIII, respectively, and inserted into PstI-HindIII site of plasmid pTV-Sig with the IgH signal sequence. The pTV-Sig is derived from the commercially available plasmid pTV118N (Takar, Tokyoa) and is inserted with the NcoI fragment of 574 bp IgH signal sequence derived from the plasmid pRSVVuERCA (Ueda, H. et al., Bio/Technology 10: 430-433, 1992) which expresses a chimeric protein of IgM and human epidermal growth factor receptor, where the HindIII site derived from PTV118N is at the 3′ side of the inserted sequence. After confirming the sequence of respective plasmids, the fragments containing IgH leader sequence and V_{H} and V_{L} sequences were cleaved with restriction enzymes NcoI and AflII, and inserted into the NcoI-AflII site of pRSVVuERCA.

Next, in order to link the variable region fragments of this plasmid containing leader sequence to the gene for the mouse erythropoietin receptor (mEpoR), the fragment extending from the IgH leader sequence to the pRSVVuERCA-derived intron downstream of V_{H}/V_{L} was amplified by PCR, and the PCR product was inserted into the EcoRI-SacII site of pBluescript™ II SK- (Stratagene). The primers used for the PCR were the oligonucleotide having the nucleotide sequence of SEQ ID No. 4 (V_{H}LeaderRiBack) and the oligonucleotide having the nucleotide sequence of SEQ ID No. 6 (CH31AS2For). After confirming respective sequences, the amplified fragments were inserted into the EcoRI-SacII site of the mEpoR expression vector pME-ER (Chiba, T. et al., Nature 362: 646-648, 1993), from which mEpoR 5′ fragment has been removed, eventually to construct plasmid vectors pME-V_{H}ER and PME-V_{L}ER.

All PCR reactions were performed using Pfu polymerase (Stratagene). Restriction enzymes and repair enzymes were by Takara Co., and the sequences were confirmed by the DNA sequencer SQ-5500 (Hitachi).

### Example 2

### Introduction of the Expression Vectors into Animal Cell and Selection of the Transfectants

Two plasmids, PME-V_{H}ER and pME-V_{L}ER, produced in Example 1, were introduced into the cell line Ba/F3 which is derived from the interleukin-3 (IL-3) dependent mouse pro-B cells, by using electroporation method. The Ba/F3 cells (2×10⁶) in the logarithmic growth period and 5 μg each of above plasmid DNAs linearized with the restriction enzyme KpnI were suspended in 100 μl HBS (Hanks' Buffered Saline: Nissui Pharmacy). Rectangular pulses, 500 volts/150 μsec, were applied 10 times using a gene introduction electric pulse generator (electrodes of 4 mm intervals, Bio-Rad). The cells was kept at room temperatures for five minutes, diluted in 4 ml RPMI 1640 culture media (Nissui Pharmacy) containing 400 μg/ml G418 (Sigma), 8 ng/ml mouse IL-3 (Genzyme) and 10% fetal bovine serum (FBS, Gibco), added into 24-well plates 1 ml each, and cultured for two weeks at 37° C. in the 5% CO₂ humidified atmosphere. The resultant colonies were mixed and cultured for another three days in the same media 100 μl cell suspensions were diluted in 2 ml RPMI 1640 medium containing 10 μg/ml hen egg lysozyme (HEL, Seikagaku) and 10% FBS (Gibco) and were subcultured. The Ba/F3 cell line is known to be released from IL-3 dependency by the ectopic expression of the EPO receptor when EPO is added to the medium.

While most cells were found to die in the subculturing, but some survived and grew. The survived cells were further subcultured in the same medium containing HEL, named Ba/HEL cells, and frozen for storage. The Ba/F3 cells to which no expression vectors were introduced did not grow in this medium.

Cloning of the Ba/HEL cells were performed by using the limiting dilution method in the 1 μg/ml HEL-containing RPMI 1640 medium (10% FBS) in which Ba/F3 cells (10⁵/ml) were also included as feeder cells. After a two-week's cultivation, the survived colonies, while the Ba/F3 cells dead, were subcultured in the same medium and frozen for storage to be used in the confirmation of protein expression and measurement of growth activity.

### Example 3

### Confirmation of Expression of Chimeric Receptor Protein in Ba/HEL Cells

The expression of chimeric protein from the introduced gene in the cloned Ba/HEL cells was confirmed by Western Blotting. The cloned cells (2×10⁶) were suspended in 20 μl lysis buffer solutions (20 mM HEPES: pH 7.5, 150 mM NaCl, 10% glycerol, 1% Triton™ X-100, 1.5 mM MgCl₂, 1.0 mM EGTA, 10 μg/ml aprotinin, 10 μg/ml leupeptin), kept for five minutes at 0° C., centrifuged for five minutes at 4° C. with 15 kg. The thus obtained supernatant of 15 μl was applied SDS polyacrylamide gel electrophoresis, and then proteins transferred from the polyacrylamide gel to a nitrocellulose membrane (Toyo Filters) using a semi-dry blotting unit (Bio-Rad) according to the manufacturer's manual. The membrane was gently shook for one hour in a TBST buffer solution (20 mM Tris-HCl: pH 7.5, 150 mM NaCl, 0.1% Tween™ 20) containing 5% skim milk for blocking, then gently shook for another hour at room temperature in a TBST buffer solution containing 1/1000 diluted rabbit anti-erythropoietin receptor intracellular domain antibody. The membrane was rinsed with the TBST buffer solution for 15 minutes at room temperature three times, probed with HRP-anti-rabbit IgG (Biosource) in TBST, and washed three times with TBST. Then the amount of peroxidase fixed on the nitrocellulose membrane was visualized on an X-ray film (Fuji RX) by the chemiluminescence technique using an ECL kit (Amersham).

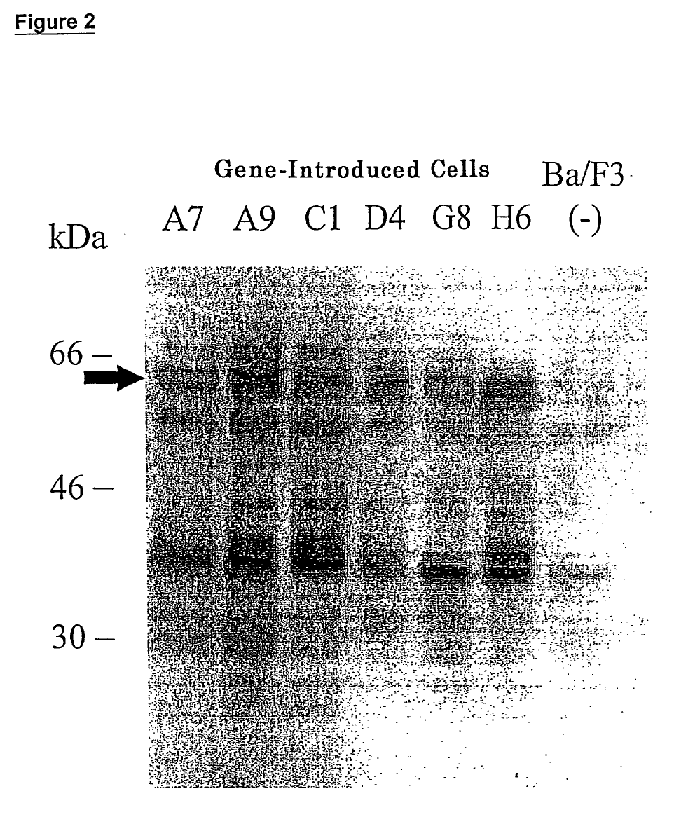

The result is shown in FIG. 2. As the figure shows, the band of about 65 to 66 kDa molecular weight was observed in the lanes for the Ba/HEL clones, while there being not in the lane for the Ba/F3 cells. In other experiments, the ˜65-66 kDa band resolved into a doublet of roughly equal intensity, which is attributed to expression of the two variable region chimeras. Twelve selected clones were all observed to express the doublet; hence expression of both the V_{H} and V_{L} constructs appear to be necessary. Bands of 40 kDa appearing in the selected clones but not the control cells may represent polypeptides truncated near the transmembrane domain.

### Example 4

### Confirmation of Antigen-Dependent Growth of Ba/HEL Cell

The clones of Ba/HEL cells were cultured with various concentrations of HEL to measure the change of the cell number over time. HEL was added in the concentrations of 0, 0.005, 0.05 and 0.5 μg/ml to the RPMI 1640 medium containing 10% FBS, seeded Ba/HEL clones with the initial concentration of 5×10³ ml, and measured the change in the number of viable cells over time by the cell count method using trypan blue.

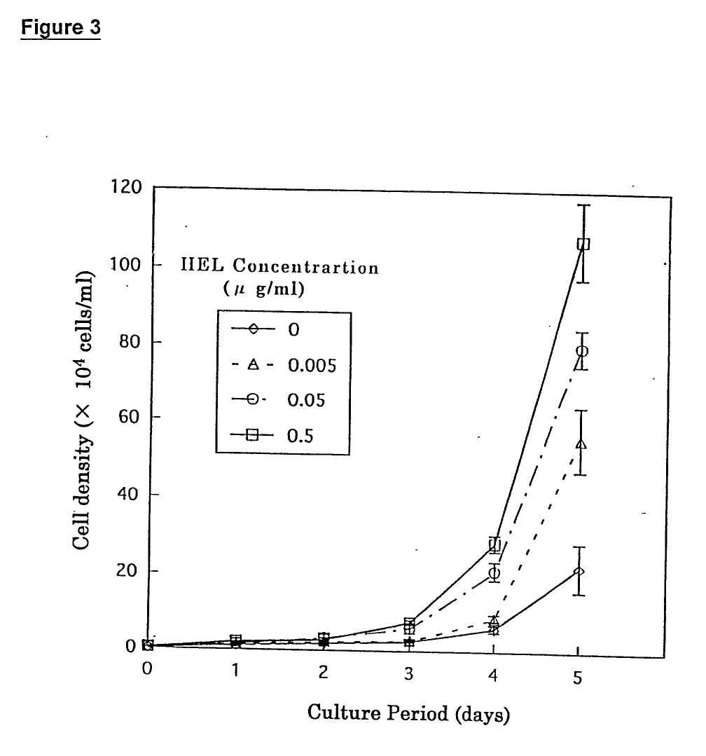

The result is shown in FIG. 3. As the figure shows, cell growth depends on the concentration of HEL in the medium. While FIG. 2 relates to the Ba/HEL cell clones G8, similar results were observed for all clones that were inspected.

Cell growth was conspicuously accelerated with as low as 5 ng/ml antigen. This concentration agrees with the detectable antigen concentration in the immunoassay based on V_{H}/V_{L} association of HyHEL-10 Fv (Ueda et al., Nature Biotech, 14, 1714-1718, 1996). Accordingly, antigen concentration can be measured, that is, immunoassay can be performed using the level of growth of this cell as an index.

### REFERENCES

1. Bach, N. L. et al. 1994. Functional expression in mast cells of chimeric receptors with antibody specificity. Cell Biophys. 24:229-236.

2. Chiba, T. et al. 1993. Tyrosine kinase activation through the extracellular domains of cytokine receptors. Nature 362:646-648.

3. Crabtree, G. and Schreiber, S. 1996. Trends Biochem. Sci. 21: 418-422.

4. D'Andrea, A. D. et al. 1989. Expression cloning of the murine erythropoietin receptor. Cell 57:277-285.

5. Davies, J. and Riechmann, L. 1994. “Camelising” human antibody fragments NMR studies on V_{H} domains. FEBS Letters 339:285-290.

6. Davies, J. and Riechmann, L. 1995. Antibody V_{H} domains as small recognition units. Bio/Technology 13:475-479.

7. Figlini, M., Marks, J. D., Winter, G., and Griffiths, A. D. 1994. In vitro assembly of repertoires of antibody chains on the surface of phage by renaturation. J. Mol. Biol. 239:68-78.

8. Glockshuber, R., Malia, M., Pfitzinger, I., and Plückthun, A. 1990. A comparison of strategies to stabilize immunoglobulin Fv-fragments. Biochemistry 29:1362-1367.

9. Hamel, P. A., Klein, M. H., Smith-Gill, S. J., and Dorrington, K. J. 1987. Relative noncovalent association constant between immunoglobulin H and L chains is unrelated to their expression of antigen-binding activity. J. Immunol. 139:3012-3020.

10. Hedrick, S. M. et al. 1993. Chimeric T cell receptor-immunoglobulin molecules: function and applications. Int. Rev. Immunol. 10:279-290.

11. Hess, S., et al. 1995. A cytotoxic CD40/p55 tumor necrosis factor receptor hybrid detects CD40 ligand on herpesvirus saimiri-transformed T cells. Eur. J. Immunol. 25:80-86.

12. Huston, J. S. et al. 1988. Protein engineering of antibody binding sites: recovery of specific activity in an antidigoxin single-chain Fv analogue produced in Escherichia coli. Proc. Natl. Acad. Sci USA 85:5879-5883.

13. Ihle, H. N., et al. 1994. Signaling by the cytokine receptor fuperfamily: JAKs and STATs. Trends Biochem. Sci. 19: 222-227.

14. Ito, K. et al. 1996. Gene Therapy, Illustrated Medicine & Science Series, Yodosha.

15. Jones, S. S. et al. 1990. Human erythropoietin receptor: cloning, expression, and biologic characterization. Blood 76:31-35.

16. Kruskal, B. A., et al. 1992. Phagocytic chimeric receptors require both transmembrane and cytoplasmic domains from the mannose receptor. J. Exp. Med. 176:1673-1680.

17. Kuramochi S. et al. 1990. Characterization of murine erythropoietin receptor genes. J. Mol. Biol. 216:567-575.

18. Lavoie, T. B., Drohan, W. N., and Smith-Gill, S. J. 1992. Experimental analysis by site-directed mutagenesis of somatic mutation effects on affinity and fine specificity in antibodies specific for lysozyme. J. Immunol. 148:503-513.

19. Leaman, D. W. et al. 1996. Regulation of STAT-dependent pathways by growth factors and cytokines. FASEB J. 10:1578-1588.

20. Lu, G. et al. 1997. Importance of the dimer-dimer interface for allosteric signal transduction and AMP cooperativity of pig kidney fructose-1,6-bisphosphatase. Site-specific mutagenesis studies of Glu-102 and Asp-1 87 residues on the 190's loop. J. Biol. Chem. 272:5076-5081.

21. Maenaka, K. et al. 1996. A stable phage-display system using a phagemid vector: phage display of hen egg-white lysozyme (HEL), Escherichia coli alkaline phosphatase, and anti-HEL monoclonal antibody, HyHEL-10. Biochem. Biophys. Res. Comm. 218:682-687.

22. Mainhart, S. G. S. J. et al. 1987. A three-dimensional model of an anti-lysozyme antibody. J. Mol. Biol. 194:713-724.

23. Makishima, F. et al. 1992. Cytotechnology, 10:15-23.

24. Miyazaki K. et al. 1994. Chemical modification and site-directed mutagenesis of Tyr36 of 3-isopropylmalate dehydrogenase from Thermus termophilus HB8. Protein Eng. 7:99-102, 1994.

25. Orlinck, J. R., et al. 1997. Requirement for cysteine-rich repeats of the Fas receptor for binding by the Fas ligand. J. Biol. Chem. 272:28889-28894.

26. Padlan, E. A. et al. 1989. Structure of an antibody-antigen complex: crystal structure of the HyHEL-10 Fab-lysozyme complex. Proc. Natl. Acad. Sci. USA 86:5938-5942.

27. Parsons H. L. et al. 1996. Directing phage selections towards specific epitopes. Protein Engineering 9:1043-1049.

28. Pharmacia Biosensor AB. 1994. BlAtechnology handbook Merk AB, Uppsala, Sweden.

29. Truong, H. T. et al. 1991. Inactive and temperature-sensitive folding mutants generated by tryptophan substitutions in the membrane-bound d-lactate dehydrogenase of Escherichia coli. Biochemistry 30:10722-10729.

30. Tsumoto, K. et al. 1994. Effect of the order of antibody variable regions on the expression of the single chain HyHEL10 Fv fragment in E. coli and the thermodynamic analysis of its antigen-binding properties. Biochem. Biophys. Res. Commun. 201:546-551.

31. Tsumoto, K. et al. 1994. Contribution to antibody-antigen interaction of structurally perturbed antigenic residues upon antibody binding. J. Biol. Chem. 269:28777-28782.

32. Tyutyulkova, S. and Paul, S. 1994. Selection of functional human immunoglobulin light chains from a phage-display library. Appl. Biochem. Biotechnol. 47:191-198.

33. Ueda, H. et al. 1992. Antigen responsive antibody-receptor kinase chimera. Bio/Technology 10:430-433.

34. Ueda, H., et al. 1993. Synthesis and expression of a DNA encoding the Fv domain of an anti-lysozyme monoclonal antibody, HyHEL-10, in Streptomyces lividans, Gene 129129-134.

35. Ueda, H. et al. 1996a. Analysis of intramolecular interaction of an antibody Fv region and its application to immunoassay [abstract]. Protein Engineering 9:819

36. Ueda, H. et al. 1996b. Open sandwich ELISA: A novel immunoassay based on the interchain interaction of antibody variable region. Nature Biotechnol. 14:1714-1718.

37. Watanabe, S., et al. 1996. Roles of the JAK-STAT system in signal transduction via cytokine receptors. Curr. Opin. Genet. Dev. 6: 587-596.

38. Wagner, C. R. et al. 1990. Site directed mutagenesis: a tool for enzyme mechanism detection. Trends Biotechnol. 1:263-270, 1990.

39. Ward, E. S. 1992 Expression and purification of antibody fragments using Escherichia coli as a host, pp. 121-138 in Antibody engineering A practical guide. Borrebaeck, C. A. K. (ed.) W. H. Freeman & Co., New York.

U.S. Patent Documents

| | | |
|---|---|---|
| U.S. Pat. No. 4,859,609 | Dull et al. | Hybrid receptors convenient |
| | | for assay |
| U.S. Pat. No. 5,030,576 | Dull et al. | Hybrid receptors convenient |
| | | for assay |
| U.S. Pat. No. 5,639,660 | Kinet et al. | Polypeptide and DNA |
| | | sequence corresponding to |
| | | the human receptor with |
| | | high affinity for IgE |
| U.S. Pat. No. 5,641,863 | Schreiber et al. | Chimeric IgG Fc receptors |
| U.S. Pat. No. 5,712,149 | Roberts | Chimeric receptor molecules |
| | | for delivery of co- |
| | | stimulatory signals |

Other Patent Documents

| | | |
|---|---|---|
| E.P. 517,895-B | Capon et al. | Chimeric chains for |
| | | receptor-associated signal |
| | | transduction pathways |
| E.P. 816,850-A | Ueda et al. | A method of measuring |
| | | antigen concentration |
| WO 95/02684 | Crabtree et al. | Regulated apoptosis |
| WO 95/02686 | Seed et al. | Redirection of cellular |
| | | immunity by receptor |
| | | chimeras |
| WO 96/23881 | Capon et al. | Chimeric receptors for |
| | (Cell Genesis) | regulating cellular |
| | | proliferation and effector |
| | | function |
| WO 96/26265 | Seed et al. | Redirection of cellular |
| | | immunity by protein |
| | | tyrosine chimeras |
| WO 97/35004 | Hawkins et al. | Cell stimulation |

Additional references can be found at various places throughout the disclosure.

### Sequence Listing

(1) GENERAL INFORMATION:

(i) APPLICANT:

Ueda, Hiroshi

Nagamune, Teruyuki

Nishimura, Hajime

Kumagai, Izumi

Tsumoto, Kouhei

Todokoro, Kazuo

Mahoney, Paula A.

Mahoney, Walt

(ii) TITLE OF INVENTION: CHIMERIC CELL-SURFACE RECEPTORS THAT UNDERGO ANTIGEN-INDUCED ASSOCIATION

(iii) NUMBER OF SEQUENCES: 6

(iv) CORRESPONDENCE ADDRESS:

(A) ADDRESSEE: MORRISON & FOERSTER LLP

(B) STREET: 755 PAGE MILL ROAD

(C) CITY: PALO ALTO

(D) STATE: CA

(E) COUNTRY: USA

(F) ZIP: 94304-1018

(v) COMPUTER READABLE FORM:

(A) MEDIUM TYPE: Diskette

(B) COMPUTER: IBM Compatible

(C) OPERATING SYSTEM: Windows

(D) SOFTWARE: FastSEQ for Windows Version 2.0b

(vi) CURRENT APPLICATION DATA:

(A) APPLICATION NUMBER: Unassigned

(B) FILING DATE: Feb. 27, 1998

(C) CLASSIFICATION:

(vii) PRIOR APPLICATION DATA:

(A) APPLICATION NUMBER: JP 9-44487

(B) FILING DATE: Feb. 27, 1997

(viii) ATTORNEY/AGENT INFORMATION:

(A) NAME: Schiff, Michael

(B) REGISTRATION NUMBER: 40,253

(C) REFERENCE/DOCKET NUMBER: 33746-20055.00

(ix) TELECOMMUNICATION INFORMATION:

(A) TELEPHONE: 650-813-5600

(B) TELEFAX: 650-494-0792

(C) TELEX: 706141

(2) INFORMATION FOR SEQ ID NO:1:

(i) SEQUENCE CHARACTERISTICS:

(A) LENGTH: 22 base pairs

(B) TYPE: nucleic acid

(C) STRANDEDNESS: single

(D) TOPOLOGY: linear

(ii) MOLECULE TYPE: Other

(xi) SEQUENCE DESCRIPTION: SEQ ID NO:1: AGGTGCACCT GCAGGAGTCT GG 22

(2) INFORMATION FOR SEQ ID NO:2:

(i) SEQUENCE CHARACTERISTICS:

(A) LENGTH: 38 base pairs

(B) TYPE: nucleic acid

(C) STRANDEDNESS: single

(D) TOPOLOGY: linear

(ii) MOLECULE TYPE: Other

(xi) SEQUENCE DESCRIPTION: SEQ ID NO:2: TTTAAGCTTA AGGACTCACC CGCCGAGACG GTGACGAG 38

(2) INFORMATION FOR SEQ ID NO:3:

(i) SEQUENCE CHARACTERISTICS:

(A) LENGTH: 27 base pairs

(B) TYPE: nucleic acid

(C) STRANDEDNESS: single

(D) TOPOLOGY: linear

(ii) MOLECULE TYPE: Other

(xi) SEQUENCE DESCRIPTION: SEQ ID NO:3: TTTCTGCAGA TCGTCCTGAC CCAGAGC 27

(2) INFORMATION FOR SEQ ID NO:4:

(i) SEQUENCE CHARACTERISTICS:

(A) LENGTH: 38 base pairs

(B) TYPE: nucleic acid

(C) STRANDEDNESS: single

(D) TOPOLOGY: linear

(ii) MOLECULE TYPE: Other

(xi) SEQUENCE DESCRIPTION: SEQ ID NO:4: TTTAAGCTTA AGGACTCACC CTTGATCTCC AGCTTGGT 38

(2) INFORMATION FOR SEQ ID NO:5:

(i) SEQUENCE CHARACTERISTICS:

(A) LENGTH: 29 base pairs

(B) TYPE: nucleic acid

(C) STRANDEDNESS: single

(D) TOPOLOGY: linear

(ii) MOLECULE TYPE: Other

(xi) SEQUENCE DESCRIPTION: SEQ ID NO:5: CCGAATTCAC CATGGGATGG AGCTGTATC 29

(2) INFORMATION FOR SEQ ID NO:6:

(i) SEQUENCE CHARACTERISTICS:

(A) LENGTH: 42 base pairs

(B) TYPE: nucleic acid

(C) STRANDEDNESS: single

(D) TOPOLOGY: linear

(ii) MOLECULE TYPE: Other

(xi) SEQUENCE DESCRIPTION: SEQ ID NO:6: CCCCGCGGGG GCGTCCAGGA GCACTGCAGT CAAGAGAACA CT 42

What is claimed as the invention is:

1. A pair of chimeric polypeptides consisting of a first polypeptide and a second polypeptide that complex with each other in the presence of an antigen, each polypeptide of the pair comprising:
an extracellular domain comprising a variable domain sequence,
a transmembrane domain, and
an effector sequence,
wherein upon expression in a host cell, the pair of chimeric polypeptides has the property that contacting the cell with an analyte promotes association of the variable domain sequence of the first polypeptide with the variable domain sequence of the second polypeptide, thereby promoting association of the effector sequence of the first polypeptide with the effector sequence of the second polypeptide.

2. A pair of chimeric polypeptides consisting of a first polypeptide and a second polypeptide that complex with each other in the presence of an antigen, each polypeptide of the pair comprising:
an extracellular domain comprising a variable domain sequence,
a transmembrane domain, and
an effector sequence,
wherein upon expression in a host cell, the pair of chimeric polypeptides has the property that contacting the cell with an analyte promotes association of the variable domain sequence of the first polypeptide with the variable domain sequence of the second polypeptide, thereby inducing a change in cell phenotype.

3. The pair of chimeric polypeptides according to claim 1, wherein the variable domain sequences of the first and second polypeptide are V_{H} and V_{L} sequences in either order.

4. The pair of chimeric polypeptides according to claim 1, wherein the antigen is lysozyme or digoxin.

5. The pair of chimeric polypeptides according to claim 1, wherein the effector sequences of the first and second polypeptide are both derived from a proliferation receptor, a degranulation receptor, a cytotoxic receptor, a phagocytic receptor, or an apoptosis receptor.

6. The pair of chimeric polypeptides according to claim 1, wherein the transmembrane domain and effector sequence of both the first and second polypeptide are derived from an erythropoietin receptor.

7. The pair of chimeric polypeptides according to claim 1, expressed in a host cell.

8. The pair of chimeric polypeptides according to claim 7, wherein contacting the host cell with the antigen results in proliferation of the host cell.

9. The pair of chimeric polypeptides according to claim 7, wherein contacting the host cell with the antigen results in degranulation of the host cell.

10. The pair of chimeric polypeptides according to claim 7, wherein contacting the host cell with the antigen results in apoptosis of the host cell.

11. The pair of chimeric polypeptides according to claim 7, wherein contacting a target cell bearing the antigen with the host cell results in cytotoxic lysis or phagocytosis of the target cell.

12. A method for obtaining the chimeric polypeptides according to claim 7, comprising the steps of:
a) introducing a host cell with an expression system for the chimeric polypeptides according to claim 1, the expression system comprising either a single expression vector encoding both the first and second polypeptide, or two expression vectors separately encoding the first and second polypeptide;
b) culturing the host cell so as to permit expression of the expression vector(s) introduced in step a);
c) contacting the host cell with the antigen;
d) selecting cells contacted with antigen that express a phenotype that depends on association of the first effector sequence with the second effector sequence.

13. A method for selecting cells expressing a polynucleotide encoding sequence, comprising the steps of:
a) introducing a host cell with an expression system for the chimeric polypeptides according to claim 1, the expression system comprising either a single expression vector encoding both the first and second polypeptide and further comprising the polynucleotide encoding sequence, or two expression vectors separately encoding the first and second polypeptide, at least one of which further comprises the polynucleotide encoding sequence;
b) culturing the host cell so as to permit expression of the expression vector(s) introduced in step a);
c) contacting the host cell with the antigen;
d) selecting cells contacted with antigen that express a phenotype that depends on association of the first effector sequence with the second effector sequence.

14. The method according to claim 13, wherein the first and second effector sequences are both sequences of a growth factor receptor, and the phenotype is increased proliferation of the cells.

15. A method for increasing the rate of proliferation of cells in a population expressing the chimeric polypeptides of claim 1, wherein the first and second effector sequences are both sequences of a growth hormone receptor, the method comprising contacting the cells with the antigen.

16. A method of reducing the number of cells in a population expressing the chimeric polypeptides of claim 1, wherein the first and second effector sequences are both sequences of an apoptosis receptor, the method comprising contacting the cells with the antigen.

17. A method for causing cytolysis or phagocytosis of a target cell bearing the antigen, comprising contacting the target cell with a cell expressing the chimeric polypeptides of claim 1, wherein the first and second effector sequences are both sequences of a cytotoxic receptor or a phagocytic receptor.

18. A method for determining an antigen in a sample, comprising preparing a reaction mixture comprising the sample and a cell expressing the chimeric polypeptides of claim 1; measuring a phenotype that depends on association of the first effector sequence with the second effector sequence; and correlating the phenotype with the presence or concentration of the antigen in the sample.
